SEQUENCE LISTING (4 sequences)

SEQ ID NO: 1 - protein, 353 aa, Homo sapiens
ASPTSPKVFPLSLCSTQPDGNVVIACLVQGFFPQEPLSVTWSESGQGVTARNFPPSQDASGDLYTTSSQLTLPATQCLAGKSVTCHVKHYTNPSQDVTVPCPVPSTPPTPSPSTPPTPSPSCCHPRLSLHRPALEDLLLGSEANLTCTLTGLRDASGVTFTWTPSSGKSAVQGPPERDLCGCYSVSSVLPGCAEPWNHGKTFTCTAAYPESKTPLTATLSKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGTCY

SEQ ID NO: 2 - protein, 10 aa, Homo sapiens
WLQGSQELPR

SEQ ID NO: 3 - DNA, 1059 bp, Homo sapiens
gcaagcttgaccagccccaaggtcttcccgctgagcctctgcagcacccagccagatgggaacgtggtcatcgcctgcctggtccagggcttcttcccccaggagccactcagtgtgacctggagcgaaagcggacagggcgtgaccgccagaaacttcccacccagccaggatgcctccggggacctgtacaccacgagcagccagctgaccctgccggccacacagtgcctagccggcaagtccgtgacatgccacgtgaagcactacacgaatcccagccaggatgtgactgtgccctgcccagttccctcaactccacctaccccatctccctcaactccacctaccccatctccctcatgctgccacccccgactgtcactgcaccgaccggccctcgaggacctgctcttaggttcagaagcgaacctcacgtgcacactgaccggcctgagagatgcctcaggtgtcaccttcacctggacgccctcaagtgggaagagcgctgttcaaggaccacctgaccgtgacctctgtggctgctacagcgtgtccagtgtcctgtcgggctgtgccgagccatggaaccatgggaagaccttcacttgcactgctgcctaccccgagtccaagaccccgctaaccgccaccctctcaaaatccggaaacacattccggcccgaggtccacctgctgccgccgccgtcggaggagctggccctgaacgagctggtgacgctgacgtgcctggcacgtggcttcagccccaaggatgtgctggttcgctggctgcaggggtcacaggagctgccccgcgagaagtacctgacttgggcatcccggcaggagcccagccagggcaccaccaccttcgctgtgaccagcatactgcgcgtggcagccgaggactggaagaagggggacaccttctcctgcatggtgggccacgaggccctgccgctggccttcacacagaagaccatcgaccgcttggcgggtaaacccacccatgtcaatgtgtctgttgtcatggcggaggtggacggcacctgctac

SEQ ID NO: 4 - DNA, 30 bp, Homo sapiens
tggctgcaggggtcacaggagctgccccgc

(19) United States
(12) Patent Application Publication
(10) Pub. No.: US 2006/0240484 A1
(43) Pub. Date: Oct. 26, 2006

(54) Protein for diagnosing diabetic retinopathy

(76) Inventors: Won Il Yoo, Gyeonggi-do (KR); Sung Ho Lee, Gyeonggi-do (KR); Kunewoo Park, Busan (KR); Yang Je Cho, Seoul (KR); Bo Young Ahn, Seoul (KR)

Correspondence Address: JHK Law, P O Box 1078, La Canada, CA, 91012-1078, US

(21) Appl. No.: 10/521,675

(22) PCT Filed: Mar. 20, 2003
(86) PCT No.: PCT/KR03/00544

§ 371 Date: Apr. 19, 2006

(30) Foreign Application Priority Data
Jul. 16, 2002 (KR) 10-2002-0041771

(51) Int. Cl. G01N 33/567; C07K 16/26

(52) U.S. Cl. 435/7.2; 530/388.25

(57) ABSTRACT

The present invention relates to material for diagnosing Diabetic retinopathy. More particularly, the present invention relates to Immunoglobulin A protein for diagnosing Diabetic retinopathy, kit for diagnosing Diabetic retinopathy comprising antibody against the protein and method for diagnosing Diabetic retinopathy. The present invention can be used as diagnosing Diabetic retinopathy.

### TECHNICAL FIELD

The present invention generally relates to diagnostic substances for diabetic retinopathy, and more specifically, to a diagnostic kit including an Immunoglobulin A protein and an antibody thereof, and a diagnostic method using the same.

### BACKGROUND OF THE INVENTION

In general, diabetes mellitus as a complex metabolic disorder causing microangiopathy is one of systemic diseases which broadly impair systemic tissues. Diabetes may affect vision, and most importantly, damage to blood vessels inside the eye (LEE Tae-hee, CHOI Young-gil. Diabetic Vascular Complications, Seoul: Koryo Medicine). Diabetic retinopathy, one of the most severe complications, becomes an important problem as life span and prevalence period of diabetic patients become longer due to improvement of living standards and development of treatment (Klein R. et al., Arch Ophthalmol. 102:520-532(1984)). Diabetic retinopathy has two stages a nonproliferative stage and a proliferative stage. The nonproliferative stage is characterized in that retinal lesions resulting from vascular disorders are limited in retina. The proliferative stage is characterized by penetration of neovascularization tissues from retina into the vitreous cavity (Green, In: Spencer W H, ed. Ophtalmic Pathology: an atlas and textbook. 4^{th} ed. Philadelphia: WB Saunder; 1124-1129 (1996)). Diabetic retinopathy is diagnosed by observation of characteristic changes in the fundus structure. Vision loss due to diabetic retinopathy results from haemorrhagia corporis vitrei and maculopathy with traction retinal detachment of yellow spot in the proliferative stage. Laser treatment with surgery treatment is well-known for its effectiveness for the vision loss (Diabetic Retinopathy Study Report Number 14: Int Ophthalmol Clin. 27:239-253(1987)). This treatment following proper steps can prevent vision loss, minimizing side effects. Diabetic retionpathy should be frequently examined and diagnosed to determine whether operation is performed on the diabetic retinopathy or not. However, since diabetic retinopathy is currently diagnosed only by funduscopy, it is difficult to detect diabetic retinopathy in its early stages. As a result, it is highly frequent for patients to miss opportunities to prevent the diabetic retinopathy and have an operation on it. Accordingly, a method is disclosed for diagnosing diabetic retinopathy easily in blood. There has been no method for diagnosing diabetic retinopathy using blood. The present inventors found a protein which varies in blood by using proteomics, and applied the protein to diagnosis. Since this protein shows a marked quantitative change between a diabetic patient with no diabetic retinopathy complication and a diabetic patient having a complication, the present invention comprising this protein is completed using accurate quantification by immunological method

### DETAILED DESCRIPTION OF THE INVENTION

In order to overcome the above-described problems, the present invention has an object to provide a useful diagnosis for diabetic retinopathy.

The present invention has another object to provide a kit for diagnosing diabetic retinopathy including the diagnosis.

The present invention has still another object to provide a method for diagnosing diabetic retinopathy.

In order to achieve the above-described objects, there is provided an immunoglobulin A protein, which is effective for diagnosing diabetic retinopathy, and a protein fragment thereof.

A sequence obtained by protein analysis corresponds to a constant site of immunoglobulin A heavy chain. The immunoglobulin A protein exists as heavy-chain and light-chain types. Since each chain has a variable region, the protein has sites having many different sequences. As a result, protein having a sequence, which may be determined as an immunoglobulin A protein, can be obtained.

The disclosed immunoglobulin A protein may have various amino acid sequences as well as SEQ ID NO:1 of heavy chain.

The amino acid sequence of H chain of Ig A is as described in SEQ ID NO:1.

The disclosed protein fragment of the immunoglobulin A can have various types of fragment including a peptide of SEQ ID NO:2.

There is provided an antibody specifically binding the protein. The antibody may be both polyclonal and monoclonal, but more preferably monoclonal.

There is also provided a kit for diagnosing diabetic retinopathy including the antibody.

The disclosed kit further comprises the antibody protein obtained by conjugating with enzyme peroxidase, alkaline phosphatase or biotin.

The rest reagents used in the disclosed diagnosis kit can be easily obtained from ingredients used in general diagnosis kits.

There is also provided a method for diagnosing diabetic retinopathy, comprising: a) treating the antibody with a blood sample and an anti-Immunoglobulin A protein conjugated with peroxidase, alkaline phosphatase or biotin; and b) measuring optical density of the compound, wherein diabetic retinopathy is diagnosed when the measured result represents below 400 mg/dL immunoglobulin A.

There are provided an Immunoglobulin A gene of SEQ ID NO:3 for coding an immunoglobulin A protein and a nucleotide of SEQ ID NO:4 for coding a peptide of SEQ ID NO:2.

Hereinafter, the present invention will be described in detail.

In the present invention, the immunoglobulin A protein of vitreous body in eyeball of diabetic retinopathy patients is shown to increase than that in healthy vitreous body. Here, the present inventor found that the protein changed in blood, that is, the immunoglobulin A protein of diabetic retinopathy patient decreases in blood than that of diabetic patients. Accordingly, a diagnosis for diabetic retinopathy is disclosed using an immunologic method.

In order to accomplish the above-described object, protein groups, which show specific changes to diabetic retinopathy, are analyzed using a proteomics method. The following results are found by analyzing quantitative changes of the proteins and the types of proteins in vitreous bodies of diabetic retinopathy patients and normal vitreous bodies. After the changes of the target protein is checked in blood, a kit for diagnosing diabetic retinopathy is prepared using a proper immunological method. First, a normal vitreous body is settled as a control group. The protein groups, which show qualitative and quantitative differences, are isolated in vitreous bodies obtained from diabetic patients and diabetic retinopathy patients by two-dimensional gel separation and image analysis. The protein groups are identified using MS and Q-TOF analyzers. The protein wherein changes were observed and identified is proved as Immunoglobulin A. Increase of Immunoglobulin A, which is hardly observed in normal vitreous body, of vitreous body in diabetic retinopathy patients was observed. However, it has not been reported that immunoglobulin A increases in vitreous body of diabetic retinopathy patients. Second, this protein showed quantitative changes in blood. When blood of diabetic patients is a control group, immunoglobulin A decreases in blood of diabetic retinopathy patients. However, this result has not been reported, either. Third, a easy, sensitive and precise method for measuring existential values of proteins is selected by preparing a kit via an immunological method.

Hereinafter, the present invention will be described in detail according to preferred embodiments.

### BRIEF DESCRIPTION OF THE DRAWINGS

FIG. 1 is a diagram illustrating a process for preprocessing vitreous body of eyeball to be applied to proteomics.

FIG. 2 shows gel pictures illustrating CBB-stained fundus vitreous body proteins after two-dimensional electrophoresis. The proteins are not showed in the marked region in a vitreous body of normal eyeball while the proteins are showed in the marked region in a vitreous body of diabetic retinopathy eyeball.

FIG. 3 shows CBB-stained gel pictures illustrating serum proteins of a diabetic retinopathy patient and a diabetic patient alone, the proteins CBB-stained after two-dimensional electrophoresis. The excessive amount of protein exists in marked region for diabetic patient alone while the decreased amount of protein be showed in the marked region for diabetic retinopathy patient.

FIG. 4 shows a graph illustrating the mass spectrum (A) of peptides treated with trypsin among proteins of the marked region of FIG. 2 using MALDI-TOF and Q-TOF analyzer, and the amino sequences of the peptide among the peptide fragments(B).

FIG. 5 shows a standard titration graph illustrating 0, 15.6, 31.25, 62.5, 125, 250, 500 ng/ml immunoglobulin A standard solution and measured optical density values after ELISA reaction.

### PREFERRED EMBODIMENTS

### EXAMPLE 1

### Sample Preparation of Vitreous Body for Analyzing Proteomics

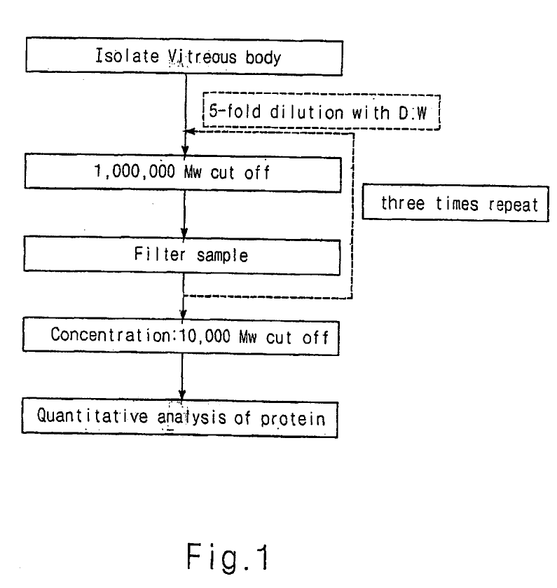

Diabetic retinopathy is one of complications resulting from long-term diabetes. Diabetic retinopathy is characterized by generation of many abnormal neovascular systems having incomplete vascular structures, which causes bleeding in vitreous body of eyeball. The bleeding results in abnormality in retina, and further weakness and loss of eyesight. In the present invention, disease indicator was searched, and information on proteins for representing disease state was obtained by analyzing proteins in vitreous bodies of a normal control group and diabetic retinopathy patients, using a proteomics method. First to apply the proteomics method to the proteins, vitreous body was treated to be easy to analyze. The vitreous body contains large amount of high molecular weight mucopolysaccharide, hyaluronic acid. However, this polysaccharide was proved to interrupt protein separation. As a result, a method was devised to remove this polysaccharide effectively (see FIG. 1). First, 4 ml vitreous body was diluted with 16 ml distilled water, and put the diluent in a tube having a cut-off membrane of 1,000,000 and centrifuged 18,000 rpm at 4° C. for 2 hours. This procedure was repeated three times to filter high molecular weight polysaccharide over 1,000,000 by difference of molecular weight. The non-filtered proteins were put in a tube having a 10,000 cut-off membrane and centrifuged 4,000 rpm, at 4° C. and then concentrated for analysis. The method for removing high molecular weight polysaccharide in the present invention enabled effective analysis by solving the problem that was not easily isolated in low pH.

### EXAMPLE 2

### Investigate of Protein Groups Changed in Vitreous Bodies of Eyeball Obtained from Normal Person and Diabetic Retinopathy Patient

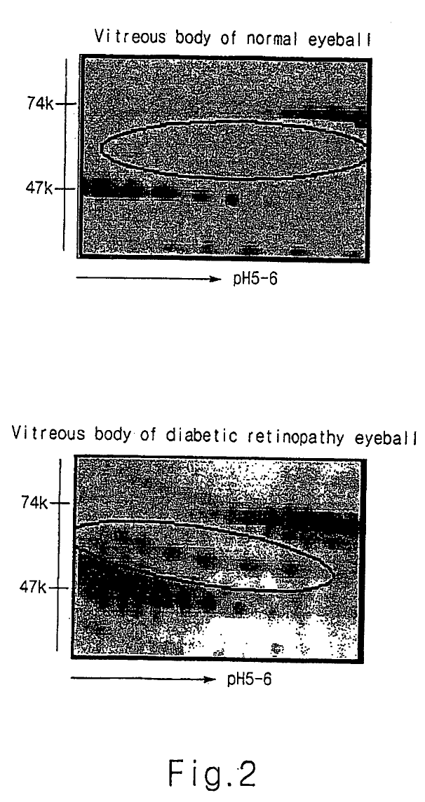

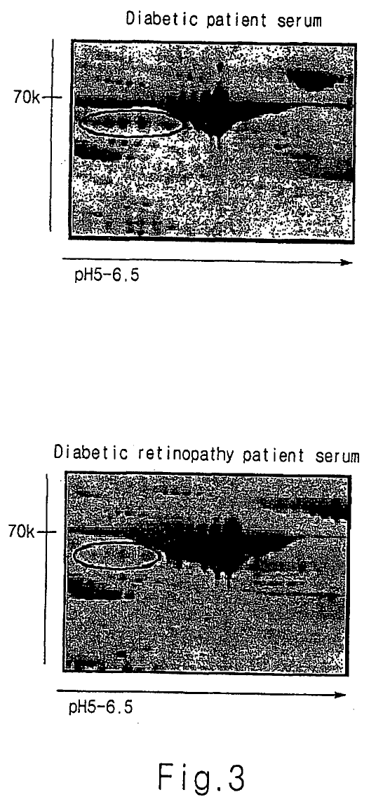

Proteins were isolated from each vitreous body and concentrated at 1 mg/mL for analysis. First, the proteins were two-dimensionally separated by a stepwise method using two different characteristics of proteins. In the first step, proteins were moved according to net charge of the proteins by applying electrical stimulus to the proteins (IEF, pH 3-10). In the second step, proteins were moved on acrylamide gel (8-18%) according to molecular weight of each protein. One-dimensional electrophoresis (protein movement according to pH) was performed on the proteins with 50 mA per gel for 12 hours. Then, two-dimensional electrophoresis (protein movement according to molecular weight) was performed on the proteins on poly-acrylamide with 50 mA per gel for 6 hours. These moved proteins were stained with a Coomaasie Brilliant Blue-G250 stain and a silver-staining method for visualizing. The difference of proteins between in normal vitreous body and in vitreous body of diabetic retinopathy patient was analyzed by using image analysis software, Phoretix (Nonlinear dynamics, UK), in computer. From analyzing the proteins in two groups, the present inventors confirmed that the protein group showing a difference existed (see FIGS. 2 and 3).

### EXAMPLE 3

### Identification of Serum Proteins that Show the Difference Between Diabetic Patient and Diabetic Retinopathy Patient

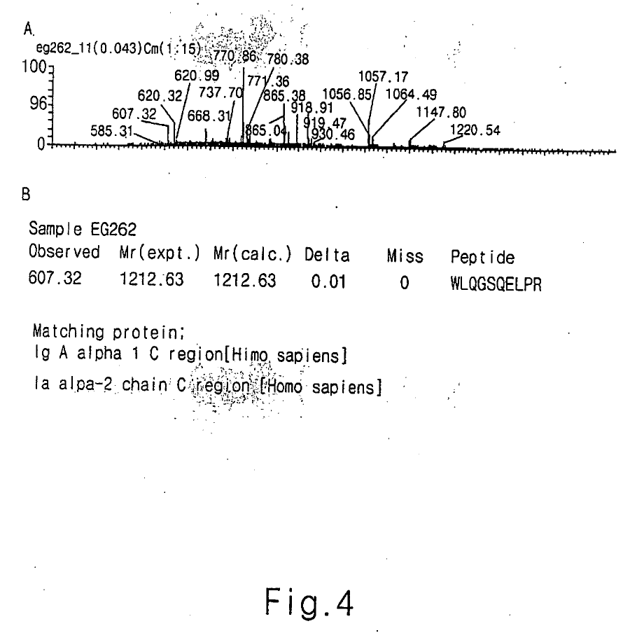

Proteins having differences in quantity and quality were searched and identified by MALDI-TOF and Q-TOF analyzers to know kinds of the proteins (see FIG. 4). It was shown that the amount of immunoglobulin A decreased in blood of diabetic retinopathy than blood of diabetic patient.

### EXAMPLE 4

### Diagnosis of Diabetic Retinopathy by Enzyme-Linked Immunosorbent Assay(ELISA)

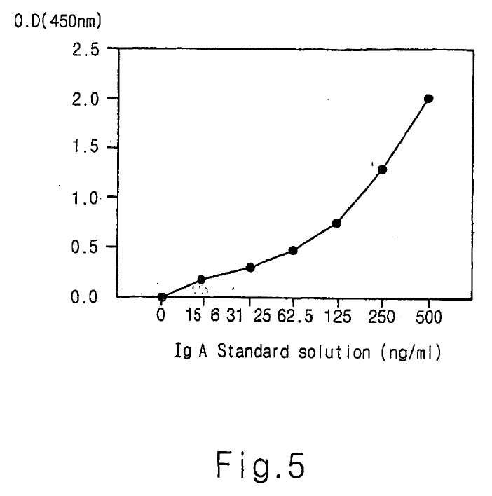

The present study was performed to find out whether serum of diabetic retinopathy among diabetic patients could be distinguished by Sandwich enzyme immunosorbent assay (ELISA) using anti-immunoglobulin A antibody. Serums were obtained from 10 normal healthy persons, 45 diabetic patients having no diabetic retinopathy and 86 diabetic retinopathy patients in hospital. First, 100 ul of anti-immunoglobulin A (Koma, Korea) (1 ug antibody protein per well; final concentration 10 ug/ml) dissolved with coating buffer (50 mM NaHCO3, pH 9.0) per well was reacted and coated in a EIA 96 well plate at room temperature for 1 hour. The each well was washed twice for 10 minutes with 400 ul PBST, and then post-coated with PBS including 1% BSA. 100 ul Serum of patients diluted with PBST buffer was put to the each well, reacted for 1 hour, and then washed five times with PBS. 100 ul of diluted peroxidase conjugated-anti-immunoglobulin A antibody (KOMA Biotech Inc., Korea) was put into the each well, and then reacted for 1 hour. After reaction, the each well was washed three times with PBS. Then, 100 ul 0.1M citrate-phosphate (pH 4.9) containing 1 mg/ml OPD (O-phenylenediamine dihydrochloride) and 0.03% H₂O₂ was put therein, and reacted at room temperature for 20-30 minutes. The reaction was stopped by 100 ul of 3M sulfuric acid, and optical density was measured at 450 nm using an ELISA reader. The amount of immunoglobulin A per blood unit volume (ml) was determined through applying conversion by standard titration curve and dilution rate to the optical density (see FIG. 5). As a result of ELISA measurement, the amount of immunoglobulin A ranged from 131.2 to 298.7 mg/dL in serum of normal person, from 226.5 to 771.9 mg/dL in serum of diabetic patient, and from 105.3 to 557.2 mg/dL in serum of diabetic retinopathy patient. These results were shown as average values in Table 1. As the measurement average value of immunoglobuline A, 217.6±82.1 mg/dL was shown in normal person, 457.6±151.6 mg/dL in diabetic patient, 244.4±117.1 mg/dL in non-proliferative diabetic retinopathy patient, and 278.6±123.6 mg/dL in proliferative diabetic retinopathy. Here, it was remarkably shown that the large amount of immunoglobulin A existed in serum of the diabetic patient group. However, it was shown that there was little difference in the amount of immunoglobulin A in serum of non-proliferative and proliferative diabetic retinopathy patient. If diabetic retinopathy was decided as positive when the amount of immunoglobulin A was below 400 mg/dL of ELISA value, 72 of 86 persons were proved as patients. Here, 83.7% of diagnostic sensitivity was shown. In case of diabetic patients without retinopathy, 22 of 45 persons were proved as patient. Here, 48.9% of diagnostic specificity was shown (see Table 2).

TABLE 1
Average value of measuring immunoglobulin A in
serum of healthy person and patient via ELISA

| | | Average 1gA Conc. (mg/dL) |
|---|---|---|
| Healthy | | 217.6 ± 82.1 |
| DM | | 457.5 ± 151.6 |
| DMR | NPDR(non- | 244.4 ± 117.1 |
| | proliferative) | |
| | PDR | 278.6 ± 123.6 |
| | (proliferative) | |

TABLE 2
Judgement of diabetic retinopathy patient via ELISA
standard 400 mg/dL (cut off)

| | | DM without | DM with |
|---|---|---|---|
| | Healthy | retinopathy | retinopathy |
| Over 400 mg/dL | 0 | 22 | 14 |
| Below 400 mg/dL | 10 | 23 | 72 |
| Total | 10 | 45 | 86 |

### INDUSTRIAL APPLICABILITY

The present invention relates to a technology for easily diagnosing diabetic retinopathy which is a complication of diabetic mellitus. There has been no effective commercial diagnostic for diabetic retinopathy. Diabetic retinopathy has been diagnosed absolutely by oculists in hospital. It is impossible for diabetic patients to diagnose diabetic retinopathy in its early stage without regular ophthalmic examination and optical defect by subjective symptoms. The present diagnostic is characterized by simple blood test, and very effective in that the development of complications can be identified before ophthalmic examination. Particularly, the present invention is advantageous in its cheap cost and simple treatment for a plurality of diabetic patients who take medical tests or consult physicians by adapting ELISA method using 96 wells which enable mass test. Also, the present invention is excellent in its accuracy and precision by using an immunochemical method. In conclusion, the present invention is effective for diagnosis of diabetic retinopathy in its early diagnosis and screening, and helpful for latent and early diabetic retinopathy patients in their decision of medication time, thereby delaying disease to severe diabetic retinopathy.

1-8. (canceled)

9. A composition for diagnosing diabetic retinopathy among diabetic mellitus patients by determining the concentration of IgA polypeptide or its fragment in blood, comprising an antibody against IgA polypeptide or its fragment.

10. The composition according to claim 9, wherein IgA polypeptide comprises the amino acid sequence described in SEQ ID NO:1.

11. The composition according to claim 9, wherein the fragment comprises the amino acid sequence described in SEQ ID NO:2.

12. A detection method for diagnosing diabetic retinopathy among diabetic mellitus patients comprising:
a) coating a solid phase with an anti-IgA antibody;
b) adding blood sample to said solid phase;
c) adding a labeled anti-IgA antibody; and
d) detecting the immunoreaction by measuring said label, wherein diabetic retinopathy is diagnosed when the measured value is lower than a predetermined value.

13. The method according to claim 12, wherein said label is a material selected from the group consisting of horseradish peroxidase, glucose-6-phoaphaste dehydrogenase, alkaline phsphatase, beta-galactosidase, fluoroisothiocyanate, rhodamine, fluorescein, luciferase, radioisotopes and particles.

14. The method according to claim 12, wherein said predetermined value is 400 mg/dL.

15. A kit for diagnosing diabetic retinopathy by determining the concentration of IgA polypeptide of its fragment in blood, comprising an antibody against IgA polypeptide or its fragment.
